SEQUENCE LISTING (2 sequences)

SEQ ID NO: 1 - protein, 27 aa, Artificial Sequence
  Synthetic
VPLPAGGGTVLTKMYPRGNHWAVGHLM

SEQ ID NO: 2 - protein, 27 aa, Artificial Sequence
  Synthetic
XXLXAGGGXVLTKXYPRGXHWAVGHLX

(19) United States
(12) Patent Application Publication
(10) Pub. No.: US 2002/0165148 A1
(43) Pub. Date: Nov. 7, 2002

(54) Analogues and derivatives of gastrin releasing peptide (GRP)

(76) Inventors: Per Franklin Nielsen, Vaerlose (DK); Ulla Ribel-Madsen, Virum (DK); Peter Andreas Nicolai Reumert Wagtmann, Rungsted Kyst (DK)

Correspondence Address: Reza Green, Esq., Novo Nordisk of North America, Inc., Suite 6400, 405 Lexington Avenue, New York, NY, 10174-6401, US

(21) Appl. No.: 09/995,989

(22) Filed: Nov. 16, 2001

Related U.S. Application Data
(60) Provisional application No. 60/253,345, filed on Nov. 28, 2000.

(30) Foreign Application Priority Data
Nov. 16, 2000 (DK) PA 2000 01720

(51) Int. Cl. A61K 38/16

(52) U.S. Cl. 514/12; 530/324

(57) ABSTRACT

The present invention relates to analogues and derivatives of GRP which have a protracted profile of action, as well as their use in treatment of e.g. Type 2 diabetes and obesity.

### CROSS REFERENCE TO RELATED APPLICATIONS

This application claims priority under 35 U.S.C. 119 of U.S. provisional application No. 60/253,345 filed on Nov. 28, 2000, and Danish application No. PA 2000 01720 filed on Nov. 16, 2000, the contents of which are fully incorporated herein by reference.

### FIELD OF THE INVENTION

The present invention relates to novel analogues and derivatives of Gastrin Releasing Peptide (GRP) which have a protracted profile of action and to methods of making and using them.

### BACKGROUND OF THE INVENTION

Peptides are widely used in medical practice, and since they can be produced by recombinant DNA technology it can be expected that their importance will increase also in the years to come. When native peptides or analogue thereof are used in therapy it is generally found that they have a high clearance. A high clearance of a therapeutic agent is inconvenient in cases where it is desired to maintain a high blood level thereof over a prolonged period of time since repeated administrations will then be necessary. Examples of peptides which have a high clearance are: ACTH, corticotropin-releasing factor, angiotensin, calcitonin, insulin, glucagon, glucagon-like peptide-1, glucagon-like peptide-2, insulin-like growth factor-1, insulin-like growth factor-2, gastric inhibitory peptide, growth hormone-releasing factor, pituitary adenylate cyclase activating peptide, secretin, enterogastrin, somatostatin, somatotropin, somatomedin, parathyroid hormone, thrombopoietin, erythropoietin, hypothalamic releasing factors, prolactin, thyroid stimulating hormones, endorphins, enkephalins, vasopressin, oxytocin, opiods and analogue thereof, superoxide dismutase, interferon, asparaginase, arginase, arginine deaminase, adenosine deaminase and ribonuclease. In some cases it is possible to influence the release profile of peptides by applying suitable pharmaceutical compositions, but this approach has various shortcomings and is not generally applicable.

Gastrin releasing peptide (GRP) is a 27 amino acid peptide which is normally produced in neuroendocrine cells of the gastrointestinal tract, lung and central nervous system. It acts by binding to a specific G-protein-coupled seven transmembrane spanning receptor and has diverse physiological effects, including stimulation of cell proliferation, hormone secretion, gastric motility, immune cell activation, and modulation of neurotransmission.

Mice with a targeted disruption of the GRP receptor have impaired blood glucose clearance and are mildly obese, indicating that GRP-signaling is important for maintaining normal glucose homeostasis and normal body weight.

It has been shown that GRP can potentiate glucose-induced insulin secretion from pancreatic islet cells in vitro.

The amino acid sequence of GRP is (SEQ. ID. No. 1):

1 2 3 4 5 6 7 8 9 10 11
Val-Pro-Leu-Pro-Ala-Gly-Gly-Gly-Thr-Val-Leu-
12 13 14 15 16 17 18 19 20 21 22
Thr-Lys-Met-Tyr-Pro-Arg-Gly-Asn-His-Trp-Ala-
23 24 25 26 27
Val-Gly-His-Leu-Met-NH₂

Unfortunately, the high clearance limits the usefulness of GRP for therapeutic purposes. Thus there still is a need for improvements in this field. Accordingly, it is an object of the present invention to provide analogues and derivatives of GRP which have a protracted profile of action relative to native GRP. It is a further object of the invention to provide analogues and derivatives of GRP which have a lower clearance than native GRP. Also, it is an object of the invention to provide a new method of treating insulin dependent and non-insulin dependent diabetes mellitus, and obesity.

### SUMMARY OF THE INVENTION

We have found that injection of GRP into diabetic rats results in a markedly accelerated clearance of blood glucose, but only for a short period of time. We have found that the short time of action of GRP in this model may be explained by rapid inactivation of GRP as a result of cleavage by the enzyme DPP IV in plasma. Furthermore, we have found that the duration of action of GRP can be extended by making GRP resistant to DPP IV-mediated degradation and this can be achieved by mutating specific amino acids at the DPP IV-cleavage site, or by acylation of the GRP, or by a combination of mutation and acylation. Such mutant or acylated analogues of GRP exhibit prolonged half-life in plasma and prolonged glucose-lowering action in vivo, making them suitable for treating conditions with elevated blood glucose levels, such as diabetes, obesity, etc.

Accordingly, the present invention relates to analogues and derivatives of GRP with the amino acid of Seq. ID No. 2:

1 2 3 4 5 6 7 8 9 10 11
Xaa-Xaa-Leu-Xaa-Ala-Gly-Gly-Gly-Xaa-Val-Leu-
12 13 14 15 16 17 18 19 20 21 22
Thr-Lys-Xaa-Tyr-Pro-Arg-Gly-Xaa-His-Trp-Ala-
23 24 25 26 27
Val-Gly-His-Leu-Xaa

wherein

Xaa at position 1 is Val or pyroglutamic acid (Pyr),

Xaa at position 2 is Pro, Gly, Val, Ile, or Thr,

Xaa at position 4 is Pro, Gly, Val, Ile, or Thr,

Xaa at position 9 is Thr or Lys,

Xaa at position 14 is Met or Leu,

Xaa at position 19 is Asn or Lys,

Xaa at position 27 is Met or Leu,

wherein the ε-amino group of one or more Lys is optionally substituted with a lipophilic substituent optionally via a spacer,

or

(a) a C-1-6-ester thereof,

(b) an amide, C-1-6-alkylamide, or C-1-6-dialkylamide thereof,

(c) an Fmoc derivative thereof, and/or

(d) a pharmaceutically acceptable salt thereof.

### DETAILED DESCRIPTION OF THE INVENTION

The present invention relates to novel analogues and derivatives of GRP. The analogues and derivatives of the invention have interesting pharmacological properties; in particular they have a more protracted profile of action than native GRP.

A simple system is used to describe analogues and derivatives of GRP. For example, Gly²-GRP designates an analogue of GRP formally derived from GRP by substituting the naturally occurring amino acid residue in position 2 (Pro) by Gly. Similarly, Lys¹³(N^{ε}-tetradecanoyl)-GRP designates a derivative of native GRP wherein the ε-amino group of the Lys residue in position 13 has been tetradecanoylated. Likewise, Lys¹³(N⁶⁸ -hexadecanoyl)-Val²-GRP designates a derivative of GRP formally derived from an analogue of GRP in which the naturally occurring amino acid residue in position 2 (Pro) is substituted by Val and wherein the _{ε}-amino group of the Lys residue in position 13 has been hexadecanoylated.

### GRP Analogues

In the present text, the designation “an analogue” and similar expressions is used to designate a peptide wherein one or more amino acid residues of the native peptide have been substituted by another amino acid residue, hence creating a mutant of the native peptide.

The total number of different amino acids between the GRP analogue and the corresponding native form of GRP does preferably not exceed five. More preferably, the number of different amino acids is four. Even more preferably, the number of different amino acids is three. Even more preferably, the number of different amino acids is two. Most preferably, the number of different amino acids is one. In order to determine the number of different amino acids, one should compare the amino acid sequence of the GRP analogue of the present invention or the amino acid sequence of the parent peptide of a GRP derivative of the present invention with native GRP. For example, there are two different amino acids between the derivative Gly⁴Leu¹⁴Lys¹³(N^{ε}-(7-deoxycholoyl))-GRP and native GRP. The differences are located at positions 4 and 14. The GRP analogues or derivatives of the present invention preferably have one or two Lys, more preferably only one Lys.

In a preferred embodiment, Xaa at position 1 is pyroglutamic acid (Pyr). In another preferred embodiment, Xaa at position 2 is Gly. In another preferred embodiment, Xaa at position 2 is Val. In another preferred embodiment, Xaa at position 4 is Gly. In another preferred embodiment, Xaa at position 4 is Val. The GRP analogue of the invention may contain any combination of the above amino acid substitutions, which effectively protects the peptide against degradation by DDP-IV.

Furthermore, Xaa at position 9 is preferably Thr, Xaa at position 14 is preferably Met, Xaa at position 19 is preferably Asn, and Xaa at position 27 is preferably Met.

Advantageously, the GRP analogue according to the invention has an amidated C-terminus, preferably —NH₂.

### Derivatives

The term “derivative” is defined as a modification of one or more amino acid residues of a peptide by chemical means, either with or without an enzyme, e.g., by alkylation, acylation, ester formation, or amide formation.

### Lipophilic Substituents

In one embodiment of the invention, the _{ε}-amino group of one more Lys can be substituted with a lipophilic substituent.

To obtain a satisfactory protracted profile of action of the GRP derivative, the lipophilic substituent attached to the GRP moiety preferably comprises 4-40 carbon atoms, in particular 8-25 carbon atoms. The lipophilic substituent may be attached to an amino group of the GRP moiety by means of a carboxyl group of the lipophilic substituent which forms an amide bond with an amino group of the amino acid residue to which it is attached.

In one preferred embodiment of the invention, the lipophilic substituent is attached to the GRP moiety by means of a spacer in such a way that a carboxyl group of the spacer forms an amide bond with an amino group of the GRP moiety. In a preferred embodiment, the spacer is an _{α,ω}-amino acid. Examples of suitable spacers are succinic acid, Lys, Glu or Asp, or a dipeptide such as Gly-Lys. When the spacer is succinic acid, one carboxyl group thereof may form an amide bond with an amino group of the amino acid residue, and the other carboxyl group thereof may form an amide bond with an amino group of the lipophilic substituent. When the spacer is Lys, Glu or Asp, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the _{ε}-amino group of Lys and the lipophilic substituent. In one preferred embodiment, such a further spacer is succinic acid which forms an amide bond with the _{ε}-amino group of Lys and with an amino group present in the lipophilic substituent. In another preferred embodiment such a further spacer is Glu or Asp which forms an amide bond with the _{ε}-amino group of Lys and another amide bond with a carboxyl group present in the lipophilic substituent, that is, the lipophilic substituent is a N^{ε}-acylated lysine residue. Other preferred spacers are N^{ε}-(γ-L-glutamyl), N^{ε}-(β-L-asparagyl), N^{ε}-glycyl, and N⁶⁸-(α-(γ-aminobutanoyl).

In another preferred embodiment of the present invention, the lipophilic substituent has a group which can be negatively charged. One preferred group which can be negatively charged is a carboxylic acid group.

In a further preferred embodiment, the lipophilic substituent comprises from 4 to 40 carbon atoms, more preferred from 8 to 25 carbon atoms.

In a further preferred embodiment, the lipophilic substituent is attached to the parent peptide by means of a spacer which is an unbranched alkane α,ω-dicarboxylic acid group having from 1 to 7 methylene groups, preferably two methylene groups which spacer forms a bridge between an amino group of the parent peptide and an amino group of the lipophilic substituent.

In a further preferred embodiment, the lipophilic substituent is attached to the parent peptide by means of a spacer which is an amino acid residue except Cys, or a dipeptide such as Gly-Lys. In the present text, the expression “a dipeptide such as Gly-Lys” is used to designate a dipeptide wherein the C-terminal amino acid residue is Lys, His or Trp, preferably Lys, and wherein the N-terminal amino acid residue is selected from the group comprising Ala, Arg, Asp, Asn, Gly, Glu, Gln, Ile, Leu, Val, Phe and Pro.

In a further preferred embodiment, the lipophilic substituent is attached to the parent peptide by means of a spacer which is an amino acid residue except Cys, or is a dipeptide such as Gly-Lys and wherein an amino group of the parent peptide forms an amide bond with a carboxylic group of the amino acid residue or dipeptide spacer, and an amino group of the amino acid residue or dipeptide spacer forms an amide bond with a carboxyl group of the lipophilic substituent.

In a further preferred embodiment, the present invention relates to a GRP derivative having a lipophilic substituent which comprises a partially or completely hydrogenated cyclopentanophenathrene skeleton.

In a further preferred embodiment, the present invention relates to a GRP derivative having a lipophilic substituent which is a straight-chain or branched alkyl group.

In a further preferred embodiment, the present invention relates to a GRP derivative having a lipophilic substituent which is the acyl group of a straight-chain or branched fatty acid.

In a further preferred embodiment, the present invention relates to a GRP derivative having a lipophilic substituent which is an acyl group selected from the group comprising CH₃(CH₂)ₙCO—, wherein n is an integer from 4 to 38, preferably an integer from 4 to 24, more preferred selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In a further preferred embodiment, the present invention relates to a GRP derivative having a lipophilic substituent which is an acyl group of a straight-chain or branched alkane α,ω-dicarboxylic acid.

In a further preferred embodiment, the present invention relates to a GRP derivative having a lipophilic substituent which is an acyl group selected from the group comprising HOOC(CH₂)ₘCO—, wherein m is an integer from 4 to 38, preferably an integer from 4 to 24, more preferred selected from the group comprising HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

In a further preferred embodiment, the present invention relates to a GRP derivative having a lipophilic substituent which is a group of the formula CH₃(CH₂)ₚ((CH₂)_{q}COOH)CHNH—CO(CH₂)₂CO—, wherein p and q are integers and p+q is an integer of from 8 to 33, preferably from 12 to 28.

In a further preferred embodiment, the present invention relates to a GRP derivative having a lipophilic substituent which is a group of the formula CH₃(CH₂)ᵣCO—NHCH(COOH)(CH₂)₂CO—, wherein r is an integer of from 10 to 24.

In a further preferred embodiment, the present invention relates to a GRP derivative having a lipophilic substituent which is a group of the formula CH₃(CH₂)ₛCO—NHCH((CH₂)₂COOH)CO—, wherein s is an integer of from 8 to 24.

In a further preferred embodiment, the present invention relates to a GRP derivative having a lipophilic substituent which is a group of the formula COOH(CH₂)ₜCO— wherein t is an integer of from 8 to 24.

In a further preferred embodiment, the present invention relates to a GRP derivative having a lipophilic substituent which is a group of the formula —NHCH(COOH)(CH₂)₄NH—CO(CH₂)ᵤCH₃, wherein u is an integer of from 8 to 18.

In a further preferred embodiment, the present invention relates to a GRP derivative having a lipophilic substituent which is a group of the formula CH₃(CH₂)ᵥCO—NH—(CH₂)_{z}—CO, wherein n is an integer of from 8 to 24 and z is an integer of from 1 to 6.

In a further preferred embodiment, the present invention relates to a GRP derivative having a lipophilic substituent which is a group of the formula —NHCH(COOH)(CH₂)₄NH—COCH((CH₂)₂COOH)NH—CO(CH₂)_{w}CH₃, wherein w is an integer of from 10 to 16.

In a further preferred embodiment, the present invention relates to a GRP derivative having a lipophilic substituent which is a group of the formula —NHCH(COOH)(CH₂)₄NH—CO(CH₂)₂CH(COOH)NH—CO(CH₂)ₓCH₃, wherein x is an integer of from 10 to 16.

In a further preferred embodiment, the present invention relates to a GRP derivative having a lipophilic substituent which is a group of the formula —NHCH(COOH)(CH₂)₄NH—CO(CH₂)₂CH(COOH)NHCO(CH₂)_{y}CH₃, wherein y is zero or an integer of from 1 to 22.

In a further preferred embodiment, the present invention relates to a GRP derivative having a lipophilic substituent which can be negatively charged. Such a lipophilic substituent can for example be a substituent which has a carboxyl group.

The lipophilic substituent is preferably characterised by having a solubility in water at 20° C. in the range from about 0.1 mg/100 ml water to about 250 mg/100 ml water, more preferable in the range from about 0.3 mg/100 ml water to about 75 mg/100 ml water. For instance, octanoic acid (C8) has a solubility in water at 20° C. of 68 mg/100 ml, decanoic acid (C10) has a solubility in water at 20° C. of 15 mg/100 ml, and octadecanoic acid (C18) has a solubility in water at 20° C. of 0.3 mg/100 ml.

In a preferred embodiment, the parent peptide of the GRP derivative is native GRP. However, the parent peptide of the insulin derivative can also be selected from any of the GRP analogues disclosed herein.

The most preferred GRP derivatives are:

1) Native GRP or analogues thereof (as described above) containing a lipophilic substituent on Lys13.

2) Lys9Arg13-GRP (with or without further amino acid substitutions as described above) containing a lipophilic substituent on Lys9.

3) Arg 13Lys19-GRP (with or without further amino acid substitution as described above) containing a lipophilic substituent on Lys19.

### Other Derivatives

The analogues or derivatives of GRP of the present invention may be in the form one or more of (a) a C-1-6-ester, (b) an amide, C-1-6-alkylamide, or C-1-6-dialkylamide, (c) an Fmoc derivative, and (d) a pharmaceutical salt. In a preferred embodiment, the GRP analogue and derivatives are in the form of an acid addition salt or a carboxylate salt, most preferably in the form of an acid addition salt.

### Preferred Specific Analogues and Derivatives of GRP According to the Invention

In a further preferred embodiment, the GRP analogue of the invention is selected from the group consisting of:

Pyr¹-GRP, Gly²-GRP, Gly⁴-GRP, Gly² Gly⁴-GRP, Val²-GRP, Val⁴-GRP, Val2Val⁴-GRP, Gly²Val⁴-GRP, and Val²Gly⁴-GRP.

In a further preferred embodiment, the GRP derivative of the invention is selected from the group consisting of:

Lys¹³(N^{ε}-dodecanoyl)-GRP, Lys¹³(N^{ε}-tetradecanoyl)-GRP, Lys¹³(N^{ε}-hexadecanoyl)-GRP, Lys¹³(N^{ε}-(γ-glutamyl(N^{α}-dodecanoyl)))-GRP, Lys¹³(N^{ε}-(γ-glutamyl(N^{α}-tetradecanoyl)))-GRP, Lys¹³(Nε-(γ-glutamyl(N^{α}-hexadecanoyl)))-GRP, Arg¹³Lys⁹(N^{ε}-dodecanoyl)-GRP, Arg¹³Lys⁹(N^{ε}-dodecanoyl)-GRP, Arg¹³Lys⁹⁻(N^{ε}-tetradecanoyl)-GRP, Arg¹³Lys⁹(N^{ε}-hexadecanoyl)-GRP, Arg¹³Lys⁹(N^{ε}-(γ-glutamyl(N^{α}-do-decanoyl)))-GRP, Arg¹³Lys⁹(N^{ε}-(γ-glutamyl(N^{α}-tetradecanoyl)))-GRP, Arg¹³Lys⁹(N^{ε}-(γ-gluta-myl(N^{α}-hexadecanoyl)))-GRP, Arg¹³Lys¹⁹(N^{ε}-dodecanoyl)-GRP, Arg¹³Lys¹⁹(N^{ε}-tetradecanoyl)-GRP, Arg¹³Lys¹⁹(N^{ε}-hexadecanoyl)-GRP, Arg¹³Lys¹⁹(N^{ε}-(γ-glutamyl(N^{α}-dodecanoyl)))-GRP, Arg¹³Lys¹⁹(N^{ε}-(γ-glutamyl(N^{α}-tetradecanoyl)))-GRP, and Arg¹³Lys¹⁹(N^{ε}-(γ-glutamyl(N⁶⁰ -hexadecanoyl)))-GRP.

### Pharmaceutical Compositions

The present invention also relates to pharmaceutical compositions comprising a GRP analogue or derivative of the present invention and a pharmaceutically acceptable vehicle or carrier.

Preferably, the pharmaceutical compositions comprise an isotonic agent, a preservative and a buffer. Examples of isotonic agents are sodium chloride, mannitol and glycerol. Examples of preservatives are phenol, m-cresol, methyl p-hydroxybenzoate and benzyl alcohol. Suitable buffers include sodium acetate and sodium phosphate.

The pharmaceutical compositions preferably further comprise a surfactant in order to improve the solubility and/or the stability of the GRP analogue or derivative.

The pharmaceutical compositions preferably also comprise zinc.

The pharmaceutical compositions preferably further comprise another antidiabetic agent. The term “antidiabetic agent” includes compounds for the treatment and/or prophylaxis of insulin resistance and diseases wherein insulin resistance is the pathophysiological mechanism.

In one embodiment of this invention, the antidiabetic agent is an insulin, more preferably human insulin.

In another embodiment of this invention, the antidiabetic agent is GLP-1(7-37) or GLP-1 (7-36)amide, or any analogue or derivative thereof, preferably a derivative disclosed in WO 98/08871 (Novo Nordisk A/S), included herein by reference.

In another embodiment the antidiabetic agent is a hypoglycaemic agent, such as an oral hypoglycaemic agent. Oral hypoglycaemic agents are preferably selected from the group consisting of DPP-IV inhibitors, sulfonylureas, biguanides, thiazolidinediones, glucosidase inhibitors, glucagon antagonists, GLP-1 agonists, potasium channel openers, insulin sensitizers, hepatic enzyme inhibitors, glucose uptake modulators, compounds modifying the lipid metabolism, compounds lowering food intake, and agents acting on the ATP-dependent potassium channel of the β-cells. Preferred sulfonylureas are tolbutamide, glibenclamide, glipizide and gliclazide. A preferred biguanide is metformin. Preferred thiazolidinediones are troglitazone and ciglitazone. A preferred glucosidase inhibitors is acarbose. Preferred agents acting on the ATP-dependent potassium channel of the β-cells are: glibenclamide, glipizide, gliclazide, and repaglinide.

The pharmaceutical compositions of the present invention may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the GRP analogue or derivative in the form of a nasal or pulmonal spray. As a still further option, the GRP analogues and derivatives of the invention can also be administered transdermally, e.g. from a patch, optionally a iontophoretic patch, or transmucosally, e.g. bucally.

The pharmaceutical compositions of the present invention may be prepared by conventional techniques, e.g. as described in Remington's Pharmaceutical Sciences, 1985 or in Remington: The Science and Practice of Pharmacy, 19^{th} edition, 1995.

For example, injectable compositions of the GRP analogue or derivative of the invention can be prepared using the conventional techniques of the pharmaceutical industry which involves dissolving and mixing the ingredients as appropriate to give the desired end product.

A composition for nasal administration of certain peptides may, for example, be prepared as described in European Patent No. 272097 (to Novo Nordisk A/S) or in WO 93/18785.

In a preferred embodiment of the present invention, the GRP analogue or derivative is provided in the form of a composition suitable for administration by injection. Such a composition can either be an injectable solution ready for use or it can be an amount of a solid composition, e.g. a lyophilised product, which has to be dissolved in a solvent before it can be injected. The injectable solution preferably contains not less than about 2 mg/ml, preferably not less than about 5 mg/ml, more preferred not less than about 10 mg/ml of the GRP analogue or derivative and, preferably, not more than about 100 mg/ml of the GRP analogue or derivative.

The particular GRP analogue or derivative to be used and the optimal dose level for any patient will depend on the disease to be treated and on a variety of factors including the efficacy of the specific peptide analogue or derivative employed, the age, body weight, physical activity, and diet of the patient, on a possible combination with other drugs, and on the severity of the case. It is recommended that the dosage of the GRP analogue or derivative of this invention be determined for each individual patient by those skilled in the art.

### Uses

The present invention also relates to the use of a GRP analogue or derivative of the invention for the preparation of a medicament which has a protracted profile of action relative to native GRP.

The present invention relates also to the use of a GRP analogue or derivative of the invention for the preparation of a medicament for the treatment of non-insulin dependent diabetes mellitus.

The present invention also relates to the use of a GRP analogue or derivative of the invention for the preparation of a medicament for the treatment of insulin dependent diabetes mellitus.

The present invention also relates to the use of a GRP analogue or derivative of the invention for the preparation of a medicament for the treatment of obesity.

The present invention also relates to the use of a GRP analogue or derivative of the invention for the preparation of a medicament with protracted effect for the prevention or treatment of Impaired Glucose Tolerance (IGT) or Impaired Fasting Glucose (IFG).

In a further preferred embodiment, the present invention relates to a method of treating any of the conditions above in a patient in need of such a treatment, comprising administering to the patient a therapeutically effective amount of a analogue or derivative of GRP and GRP analogue of the present invention together with a pharmaceutically acceptable carrier.

The patient is preferably a mammal, more preferably a human.

### Methods of Production

The parent peptide can be produced by a method which comprises culturing a host cell containing a DNA sequence encoding the polypeptide and capable of expressing the polypeptide in a suitable nutrient medium under conditions permitting the expression of the peptide, after which the resulting peptide is recovered from the culture.

The medium used to culture the cells may be any conventional medium suitable for growing the host cells, such as minimal or complex media containing appropriate supplements. Suitable media are available from commercial suppliers or may be prepared of published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of peptide in question.

The DNA sequence encoding the parent peptide may suitably be of genomic or cDNA origin, for instance obtained by preparing a genomic or cDNA library and screening for DNA sequences coding for all or part of the peptide by hybridisation using synthetic oligonucleo-tide probes in accordance with standard techniques (see, for example, Sambrook, J, Fritsch, EF and Maniatis, T, Molecular Cloning: A Laboratory Manual, Cold Spring Harbor Laboratory Press, New York, 1989). The DNA sequence encoding the peptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. The DNA sequence may also be prepared by polymerase chain reaction using specific primers, for instance as described in U.S. Pat. No. 4,683,202 or Saiki et al., Science 239 (1988), 487-491.

The DNA sequence may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the peptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide of the invention in a variety of host cells are well known in the art, cf. for instance Sambrook et al., supra.

The DNA sequence encoding the peptide may also, if necessary, be operably connected to a suitable terminator, polyadenylation signals, transcriptional enhancer sequences, and translational enhancer sequences. The recombinant vector of the invention may further comprise a DNA sequence enabling the vector to replicate in the host cell in question.

The vector may also comprise a selectable marker, e.g. a gene the product of which complements a defect in the host cell or one which confers resistance to a drug, e.g. ampicillin, kanamycin, tetracyclin, chloramphenicol, neomycin, hygromycin or methotrexate.

To direct a parent peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., supra).

The host cell into which the DNA sequence or the recombinant vector is introduced may be any cell which is capable of producing the present peptide and includes bacteria, yeast, fungi and higher eukaryotic cells. Examples of suitable host cells well known and used in the art are, without limitation, E. coli, Saccharomyces cerevisiae, or mammalian BHK or CHO cell lines.

Introduction of a lipophilic substituent onto a parent peptide (GRP or GRP analogue) can be obtained by the following general method: 1 equivalent of parent peptide is dissolved in water to a concentration of 1-50 mg peptide per ml H₂O, and diluted by N-Methyl-2-pyrrolidone (NMP) to the ratio 4:1. Then, 1-10 equivalents of an ONSu ester (2,5-dioxopyrrolidin-1-yl ester) of the lipophilic group (e.g. tetradecanoic acid 2,5-dioxopyrrolidin-1-yl ester.) dissolved in NMP is added, followed by addition of 1-10 equivalents of a tertiary amine, e.g. DIEA. The reaction mixture is allowed to react for 2-20 hours.

Furthermore, the lipophilic substituent can be introduced onto the parent peptide by the any of the acylation methods disclosed in WO 00/55119, which are included herein by reference.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### Biological Testing

### Protraction of GRP Analogues and Derivatives after s.c. Administration

The protraction of GRP analogues and derivatives of the invention can be determined by monitoring the concentration thereof in plasma after sc administration to healthy pigs, using the method described below. For comparison also the concentration in plasma of native GRP after sc. administration is followed.

Pigs (50% Duroc, 25% Yorkshire, 25% Danish Landrace, app 40 kg) are fasted from the beginning of the experiment. To each pig 0.5 nmol of test compound per kg body weight is administered in a 50 μM isotonic solution (5 mM phosphate, pH 7.4, 0.02% Tween®-20 (Merck), 45 mg/ml mannitol (pyrogen free, Novo Nordisk). Blood samples are drawn from a catheter in vena jugularis at different hours. 5 ml of the blood samples are poured into chilled glasses containing 175 μl of the following solution: 0.18 M EDTA, 1500 KIE/ml aprotinin (Novo Nordisk) and 3% bacitracin (Sigma), pH 7.4. Within 30 min, the samples are centrifuged for 10 min at 5-6000*g. Temperature is kept at 4° C. The supernatant is pipetted into different glasses and kept at minus 20° C. until use.

The plasma concentrations of the peptides are determined by RIA using an anti-body specific for a region of GRP. The entire procedure is carried out at 4° C.

The assay is carried out as follows: 100 μl plasma is mixed with 271 μl 96% ethanol, mixed using a vortex mixer and centrifuged at 2600*g for 30 min. The supernatant is decanted into Minisorp tubes and evaporated completely (Savant Speedvac AS290). The evaporation residue is reconstituted in the assay buffer consisting of 80 mM NaH₂PO₄/Na₂HPO₄, 0.1 % HSA (Orpha 20/21, Behring), 10 mM EDTA, 0.6 mM thiomersal (Sigma), pH 7.5. Samples are reconstituted in volumes suitable for their expected concentrations, and are allowed to reconstitute for 30 min. To 300 μl sample, 100 μl antibody solution in dilution buffer containing 40 mM NaH₂PO₄/Na₂HPO₄, 0.1 % HSA, 0.6 mM thiomersal, pH 7.5, was added. A non-specific sample is prepared by mixing 300 μl buffer with 100 μl dilution buffer. Individual standards are prepared from freeze dried stocks, dissolved in 300 μl assay buffer. All samples are pre-incubated in Minisorp tubes with antibody as described above for 72 h. 200 μl tracer in dilution buffer containing 6-7000 CPM is added, samples are mixed and incubated for 48 h. 1.5 ml of a suspension of 200 ml per liter of heparin-stabilised bovine plasma and 18 g per liter of activated carbon (Merck) in 40 mM NaH₂PO₄/Na₂HPO₄, 0.6 mM thiomersal, pH 7.5, is added to each tube. Before use, the suspension is mixed and allowed to stand for 2 h at 4° C. All samples are incubated for 1 h at 4° C. and then centrifuged at 3400*g for 25 min. Immediately after the centrifugation, the supernatant is decanted and counted in a ny-counter. The concentration in the samples is calculated from individual standard curves.

1. An analogue or derivative of gastrin releasing protein (GRP) having Seq ID No. 2:
```
  1 2 3 4 5 6 7 8 9 10 11
Xaa-Xaa-Leu-Xaa-Ala-Gly-Gly-Gly-Xaa-Val-Leu-
  12 13 14 15 16 17 18 19 20 21 22
Thr-Lys-Xaa-Tyr-Pro-Arg-Gly-Xaa-His-Trp-Ala-
  23 24 25 26 27
Val-Gly-His-Leu-Xaa
```
wherein
Xaa at position 1 is Val or pyroglutamic acid (Pyr),
Xaa at position 2 is Pro, Gly, Val, Ile, or Thr,
Xaa at position 4 is Pro, Gly, Val, lie, or Thr,
Xaa at position 9 is Thr or Lys,
Xaa at position 14 is Met or Leu,
Xaa at position 19 is Asn or Lys,
Xaa at position 27 is Met or Leu,
wherein the ε-amino group of one or more Lys is optionally substituted with a lipophilic substituent optionally via a spacer,
or
(a) a C-1-6-ester thereof,
(b) an amide, C-1-6-alkylamide, or C-1-6-dialkylamide thereof,
(c) an Fmoc derivative thereof, and/or
(d) a pharmaceutically acceptable salt thereof,
except a peptide with the amino acid sequence having Seq. ID. No. 1 Val-Pro-Leu-Pro-Ala-Gly-Gly-Gly-Thr-Val-Leu-Thr-Lys-Met-Tyr-Pro-Arg-Gly-Asn-His-Trp-Ala-Val-Gly-His-Leu-Met

2. The analogue of gastrin releasing peptide (GRP) according to claim 1, wherein Xaa at position 1 is pyroglutamic acid (Pyr).

3. The analogue of gastrin releasing peptide (GRP) according to claim 1, wherein Xaa at position 2 is Gly.

4. The analogue of gastrin releasing peptide (GRP) according to claim 1, wherein Xaa at position 2 is Val.

5. The analogue of gastrin releasing peptide (GRP) according to claim 1, wherein Xaa at position 4 is Gly.

6. The analogue of gastrin releasing peptide (GRP) according to claim 1, wherein Xaa at position 4 is Val.

7. The analogue of gastrin releasing peptide (GRP) according to claim 2, wherein the total number of different amino acids between the gastrin releasing peptide (GRP) analogue and native gastrin releasing peptide (GRP) is five.

8. The analogue of gastrin releasing peptide (GRP) according to claim 2, wherein the total number of different amino acids between the gastrin releasing peptide (GRP) analogue and native gastrin releasing peptide (GRP) is four.

9. The analogue of gastrin releasing peptide (GRP) according to claim 2, wherein the total number of different amino acids between the gastrin releasing peptide (GRP) analogue and native GRP is three.

10. The analogue of gastrin releasing peptide (GRP) according to claim 2, wherein the total number of different amino acids between the gastrin releasing peptide (GRP) analogue and native gastrin releasing peptide (GRP) is two.

11. The analogue of gastrin releasing peptide (GRP) according to claim 2, wherein the total number of different amino acids between the gastrin releasing peptide (GRP) analogue and native gastrin releasing peptide (GRP) is one.

12. The analogue of gastrin releasing peptide (GRP) according to claim 1, wherein Xaa at position 9 is Thr.

13. The analogue of gastrin releasing peptide (GRP) according to claim 1, wherein Xaa at position 14 is Met.

14. The analogue of gastrin releasing peptide (GRP) according to claim 1, wherein Xaa at position 19 is Asn.

15. The analogue of gastrin releasing peptide (GRP) according to claim 1, wherein Xaa at position 27 is Met.

16. The analogue of gastrin releasing peptide (GRP) according to claim 1 having an amidated C-terminus, preferably —NH₂.

17. The derivative of gastrin releasing peptide (GRP) according to claim 1, wherein the ε-amino group of one or more Lys is substituted with a lipophilic substituent optionally including a spacer.

18. The derivative of gastrin releasing peptide (GRP) according to claim 17, wherein one or two Lys is substituted with a lipophilic substituent optionally via a spacer.

19. The derivative of gastrin releasing peptide (GRP) according to claim 18, wherein only one Lys is substituted with a lipophilic substituent optionally via a spacer.

20. The derivative of gastrin releasing peptide (GRP) according to claim 17, wherein the lipophilic substituent comprises from 4 to 40 carbon atoms, more preferred from 8 to 25 carbon atoms.

21. The derivative of gastrin releasing peptide (GRP) according to claim 17, wherein the lipophilic substituent comprises from 8 to 25 carbon atoms.

22. The derivative of gastrin releasing peptide (GRP) according to claim 17, wherein a lipophilic substituent is attached to an amino acid residue in such a way that a carboxyl group of the lipophilic substituent forms an amide bond with the ε-amino group of Lys.

23. The derivative of gastrin releasing peptide (GRP) according to claim 17, wherein the lipophilic substituent is attached to the parent peptide by means of a spacer.

24. The derivative of gastrin releasing peptide (GRP) according to claim 22, wherein the spacer is an unbranched alkane a,co-dicarboxylic acid group having from 1 to 7 methylene groups, preferably two methylene groups, which form a bridge between an amino group of the parent peptide and an amino group of the lipophilic substituent.

25. The derivative of gastrin releasing peptide (GRP) according to claim 22, wherein the spacer is an unbranched alkane α,ω-dicarboxylic acid group having two methylene groups, which form a bridge between an amino group of the parent peptide and an amino group of the lipophilic substituent.

26. The derivative of gastrin releasing peptide (GRP) according to claim 22, wherein the spacer is an amino acid residue except Cys, or a dipeptide such as Gly-Lys.

27. The derivative of gastrin releasing peptide (GRP) according to claim 24, wherein the ε-amino group of Lys forms an amide bond with a carboxylic group of the amino acid residue or dipeptide spacer, and an amino group of the amino acid residue or dipeptide spacer forms an amide bond with a carboxyl group of the lipophilic substituent.

28. The derivative of gastrin releasing peptide (GRP) according to claim 17, wherein the lipophilic substituent comprises a partially or completely hydrogenated cyclopentanophenathrene skeleton.

29. The derivative of gastrin releasing peptide (GRP) according to claim 17, wherein the lipophilic substituent is a straight-chain or branched alkyl group.

30. The derivative of gastrin releasing peptide (GRP) according to claim 17, wherein the lipophilic substituent is the acyl group of a straight-chain or branched fatty acid.

31. The derivative of gastrin releasing peptide (GRP) according to claim 28, wherein the acyl group is selected from the group comprising CH₃(CH₂)ₙCO—, wherein n is 4 to 38.

32. The derivative of gastrin releasing peptide (GRP) according to claim 28, wherein the acyl group is selected from the group comprising CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₂₀CO— and CH₃(CH₂)₂₂CO—.

33. The derivative of gastrin releasing peptide (GRP) according to claim 17, wherein the lipophilic substituent is an acyl group of a straight-chain or branched alkane α,ω-dicarboxylic acid.

34. The derivative of gastrin releasing peptide (GRP) according to claim 30, wherein the acyl group is selected from the group comprising HOOC(CH₂)ₘCO—, wherein m is from 4 to 38.

35. The derivative of gastrin releasing peptide (GRP) according to claim 30, wherein the acyl group is selected from the group comprising HOOC(CH₂)ₘCO—, wherein m is from 4 to 24.

36. The derivative of gastrin releasing peptide (GRP) according to claim 30, wherein the acyl group is selected from the group comprising HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

37. The derivative of gastrin releasing peptide (GRP) according to claim 17, wherein the lipophilic substituent is a group of the formula CH₃(CH₂)ₚ((CH₂)_{q}COOH)CHNH—CO(CH₂)₂CO—, wherein p and q are integers and p+q is an integer of from 8 to 33.

38. The derivative of gastrin releasing peptide (GRP) according to claim 17, wherein the lipophilic substituent is a group of the formula CH₃(CH₂)ₚ((CH₂)_{q}COOH)CHNH—CO(CH₂)₂CO—, wherein p and q are integers and p+q is an integer of from 12 to 28.

39. The derivative of gastrin releasing peptide (GRP) according to claim 17, wherein the lipophilic substituent is a group of the formula CH₃(CH₂)ᵣCO—NHCH(COOH)(CH₂)₂CO— wherein r is an integer of from 10 to 24.

40. The derivative of gastrin releasing peptide (GRP) according to claim 17, wherein the lipophilic substituent is a group of the formula CH₃(CH₂)ₛCO—NHCH((CH₂)₂COOH)CO—, wherein s is an integer of from 8 to 24.

41. The derivative of gastrin releasing peptide (GRP) according to claim 17, wherein the lipophilic substituent is a group of the formula —NHCH(COOH)(CH₂)₄NH—CO(CH₂)ᵤCH₃, wherein u is an integer of from 8 to 18.

42. The derivative of gastrin releasing peptide (GRP) according to claim 17, wherein the lipophilic substituent is a group of the formula —NHCH(COOH)(CH₂)₄NH—COCH((CH₂)₂COOH)NH—CO(CH₂)_{w}CH₃, wherein w is an integer of from 10 to 16.

43. The derivative of gastrin releasing peptide (GRP) according to claim 17, wherein the lipophilic substituent is a group of the formula —NHCH(COOH)(CH₂)₄NH—CO(CH₂)₂CH(COOH)NH—CO(CH₂)ₓCH₃, wherein x is an integer of from 10 to 16.

44. The derivative of gastrin releasing peptide (GRP) according to claim 17, wherein the lipophilic substituent is a group of the formula —NHCH(COOH)(CH₂)₄NH—CO(CH₂)₂CH(COOH)NH—CO(CH₂)_{y}CH₃, wherein y is zero or an integer of from 1 to 22.

45. A pharmaceutical composition comprising the analogue or derivative of gastrin releasing peptide (GRP) according to claim 1 and a pharmaceutically acceptable vehicle or carrier.

46. The pharmaceutical composition of claim 40, further comprising another antidiabetic agent.

47. The pharmaceutical composition of claim 41, wherein the antidiabetic agent is insulin.

48. The pharmaceutical composition of claim 41, wherein the antidiabetic agent is a hypoglycaemic agent.

49. The pharmaceutical composition of claim 41, wherein the antidiabetic agent is GLP-1 or an analogue or derivative thereof.
